(19) United States
(12) Patent Application Publication
(10) Pub. No.: US 2026/0315513 A1
(43) Pub. Date: Oct. 15, 2026

(54) METHOD FOR PRODUCING THERAPEUTIC RNA PLATELET VEHICLES

(71) Applicant: HEMOSTOD SA, Saint-Sulpice (CH)

(72) Inventors: Laurent BURNIER, Lausanne (CH); Magali HUMBERT, Romont (CH); Céline ROCCA, Ecublens (CH)

(21) Appl. No.: 19/482,701

(22) PCT Filed: May 6, 2024
(86) PCT No.: PCT/EP2024/062458

§ 371 Date: Nov. 7, 2025

(30) Foreign Application Priority Data
May 7, 2023 (EP) 23171968.3

(51) Int. Cl. A61K 31/7105; A61K 35/19; A61K 48/00; C07K 14/705; C12N 5/078; C12N 15/85

(52) U.S. Cl. CPC A61K 31/7105; A61K 35/19; A61K 48/0058; C07K 14/705; C12N 5/0644; C12N 15/85; C12N 2830/34; C12N 2830/85

(57) ABSTRACT

The present invention relates to a method for producing therapeutic RNA platelet vehicles, including transducing a therapeutic RNA sequence together with a therapeutic RNA packaging sequence including an RNA binding protein sequence into progenitors of megakaryocytes and/or CD34-expressing hematopoietic stem and progenitor cells (HSPC), immature megakaryocytes, and/or megakaryocytes, using a DNA-based vector, the therapeutic RNA sequence and/or packaging sequence encoding a therapeutic RNA product; activating expression of the therapeutic RNA sequence and/or packaging sequence; and generating platelets from mature megakaryocytes derived from the transduced progenitors of megakaryocytes and/or from the transduced CD34-expressing HSPC and/or from the transduced immature megakaryocytes and/or from the transduced megakaryocytes, wherein said expression activation is regulated to occur during maturation towards megakaryocytes and/or platelet formation, the generated platelet vehicles are functionally reactive to activation with specific agonists, the generated platelet vehicles are activated by extracellular signals, and wherein the generated platelet vehicles contain the therapeutic RNA product.

### CROSS REFERENCE TO RELATED APPLICATIONS

The present application is a U.S. National Phase of International Application No. PCT/EP2024/062458 entitled “METHOD FOR PRODUCING THERAPEUTIC RNA PLATELET VEHICLES,” and filed on May 6, 2024. International Application No. PCT/EP2024/062458 claims priority to European Patent Application No. 23171968.3 filed on May 7, 2023. The entire contents of each of the above-listed applications are hereby incorporated by reference for all purposes.

### SEQUENCE LISTING

The material in the accompanying sequence listing is hereby incorporated by reference in its entirety into this application. The accompanying file, named SequenceListing_DTS25316PCTUS was created on Nov. 7, 2025 and is 9 KB.

### TECHNICAL FIELD

The present invention belongs to the technical field of biotechnological techniques for producing RNA platelet vehicles, in particular for therapeutic purposes.

The present invention provides a method for producing therapeutic RNA platelet vehicles to enable safe delivery of a therapeutic RNA sequence to a specific target tissue and/or cells of a patient in need thereof, in particular for the treatment of a disease such as cancer, acute inflammation, chronic inflammation, liver disease, bleeding disorder, and/or coagulation disorder.

The invention further provides a therapeutic RNA platelet vehicle engineered through the above-described method, and therapeutic uses thereof.

Still further, the invention provides a pharmaceutical composition comprising therapeutic RNA platelet vehicles engineered through the above-described method, and therapeutic uses thereof.

### TECHNICAL BACKGROUND

Delivery of pharmaceutical compounds (also referred to as therapeutic compounds or therapeutic products) to a specific cell, tissue, or organ in the body of a patient is until now a challenge.

Plasmids, antisense oligonucleotides (ASO) and new drug modalities, such as small interfering RNA (siRNA) and microRNA (miRNA), gene-editing guide RNA (gRNA), long non-coding RNA (lncRNA), and small activating RNA (saRNA), have a promising potential for use in treatment of many pathological conditions, but still face the challenge of delivery to target human tissues and/or cells.

Besides the challenge of administration, half-life in plasma, and proper distribution to the pharmacological target, these compounds are biologically active, therefore limiting the therapeutic dosage to avoid toxicity and off-target effects.

New drug delivery systems, such as lipid nanoparticles or exosomes, suffer from the same problems in terms of intrinsic toxicity, systemic distribution, and manufacturing challenges.

Therefore, safe targeted drug delivery is still a challenge.

With the emergence of new drug modalities and new classes of targets, safe tissue-specific delivery became of utmost importance.

Platelets are small, anucleated cells that circulate in the blood and are involved in hemostasis.

Platelets have been proposed as vehicles for delivery of therapeutics to a target site of interest within the body of a patient, for disease treatment.

Platelets are capable of releasing their cargo, protected within their intracellular granules, such as alpha-granules, dense granules, and lysosomes, only when activated. Each granule contains a respective, distinct cargo, with different biogenesis. Moreover, platelets generate microparticles upon strong activation (Burnier et al., 2009). These microparticles comprise exosomes and ectosomes, and differ by their cargo and mechanism of formation. Circulating platelets are recruited at the inflamed vasculature where they are activated, and release granules and/or microparticle content. Altogether, platelets possess the full machinery to immediately deliver their cargo at very specific target sites, such as inflammation or bleeding sites.

Thus, using functional platelets to deliver therapeutics is elegant and allows for controlled, target-specific release of therapeutics at a site of interest.

Passive loading of therapeutics inside or at the surface of platelets necessitates specific cell culture media and process to induce or force loading, often jeopardizing quality of both the loaded therapeutic and the platelets themselves.

Low quality platelets result in low platelet activation, in turn resulting in low granules and microvesicles release. Therefore, the cargo that is loaded into an intracellular compartment, will not be successfully released.

WO2020/006539A1 discloses methods for preparing drug-loaded platelets, wherein platelets are treated with a drug and a loading buffer. This means that platelets are passively loaded, with the risk of impairing their functionality.

WO2021231990A1 disclosed a method for producing an engineered megakaryocyte or progenitor with reduced thrombogenic potential and/or producing a platelet with a reduced thrombogenic potential by deletion of at least one gene involved in the recognition of primary stimuli of thrombus formation and recognition of the secondary mediators of thrombus formation and in the release of secondary mediators of thrombus formation. Here, manipulation of membrane proteins may result in a non-predictive change of cellular membrane behavior and physic due to lipid composition modification (Meyer, Venturoli and Smit, 2008; van Uitert, Le Gac and van den Berg, 2010; Parton, Klingelhoefer and Sansom, 2011; Dunton et al., 2014; Fowler et al., 2016). Furthermore, inhibition of platelet thrombogenic potential may result in reduced physiological activation of platelets at the inflammation site.

One major aspect that still remains unanswered is how to implement efficient loading of platelet vesicles with therapeutic RNA.

So far, several RNA binding proteins have been described to localize within vesicles (Table 1).

Therefore, the RNA binding protein will recognize a specific sequence within the RNA, and tether the RNA to the vesicles.

While platelets can be activated in presence of specific agonists, one can improve this response using wild type activator receptors, and modulate them to improve in situ activation.

Triggering receptors expressed on myeloid cells (TREMs) family are immune receptors that are broadly expressed on myeloid cells, and encoded in a gene cluster at the chromosome 6p21.1 that includes NCR2 (encoding NKp44), TREM1, TREML4 (encoding TREM-like 4), TREML2, TREM2 and TREML1.

TREML1, also known as TLT-1, is exclusively expressed in platelets and megakaryocytes (Coxon, Geer and Senis, 2017) and localizes to the alpha-granules (Washington et al., 2004; Smith et al., 2018).

Upon platelet activation, TREML1 surface expression increases rapidly (Smith et al., 2018).

TREML1 binds to fibrinogen and thus participates to the platelet aggregation (Washington et al., 2009). TREML1 does not associate with DAP12, but has an extended cytoplasmic domain containing two tyrosine motifs resembling immunoreceptor tyrosine-based inhibitory motifs (Washington, Quigley and McVicar, 2002).

One motif was shown to recruit the protein tyrosine phosphatase SHP2 (Barrow et al., 2004), which can mediate either activation or inhibition, depending on context. In the case of TREML1, recruitment of SHP2 was associated with intracellular calcium release.

The transmembrane protein family Notch mediates both cell-cell interactions and extracellular signaling within the cell.

There are 4 NOTCH receptors (NOTCH1-4) with a high homology structurally and across species.

NOTCH proteins consist on an extracellular cell domain (ECD), an Epidermal Growth Factor (EGF) like repeats, binding to the ligands and an intracellular domain (ICD), containing an RBP-Jkappa-associated module domain (RAM) and seven Ankyrin repeats essential for signal transduction, two nuclear localization signals (NLS), a transactivation domain and a proline-glutamate-serine-threonine rich (PEST) domain that regulates the stability of NOTCH.

In platelets, NOTCH1 is expressed at the surface and, upon thrombin activation, leads to an activation cascade including extracellular vesicles secretion via the PI3K-AKT pathway (Chaurasia et al., 2022).

In the light of the above, it is an object of the present invention to provide a method for producing therapeutic RNA platelet vehicles allowing obtaining therapeutic RNA platelets vehicles that are capable of delivering target-specific treatment to a target site (e.g. cells and/or tissue) within the body of a patient in need thereof, while protecting a therapeutic RNA sequence from degradation or inhibition, and avoiding passive loading of platelets.

The object is solved according to the present invention by a method for producing therapeutic RNA platelet vehicles.

The present invention provides a method for producing therapeutic RNA platelet vehicles, the method comprising at least the following steps of:
S1: transducing at least one therapeutic RNA sequence together with at least one therapeutic RNA packaging sequence including an RNA binding protein sequence into progenitors of megakaryocytes and/or CD34-expressing hematopoietic stem and progenitor cells (HSPC), immature megakaryocytes, and/or megakaryocytes, using a DNA-based vector, the therapeutic RNA sequence and/or packaging sequence encoding at least one therapeutic RNA product;
S2: activating the expression of the therapeutic RNA sequence and/or the therapeutic RNA packaging sequence, and
S3: generating platelets from mature megakaryocytes derived from the transduced progenitors of megakaryocytes and/or from the transduced CD34-expressing hematopoietic stem and progenitor cells (HSPC), and/or from the transduced immature megakaryocytes and/or from the transduced megakaryocytes,
wherein said expression activation step is regulated to occur during maturation towards megakaryocytes and/or platelet formation;
wherein the generated platelet vehicles are functionally reactive to activation with specific agonists;
wherein the generated platelet vehicles are activated by extracellular signals, and
wherein the generated platelet vehicles contain the at least one therapeutic RNA product.

The present invention provides a method for producing therapeutic RNA platelet vehicles.

In particular, therapeutic RNA platelet vehicles engineered through the method can be used for treatment, preferably target-specific treatment, of a disease such as cancer, acute inflammation, chronic inflammation, bleeding disorder, coagulation disorder, and/or liver disease.

Also, therapeutic RNA platelet vehicles engineered through the method of the invention can be used for the preparation of a pharmaceutical composition for treatment, preferably target-specific treatment, of a disease such as cancer, acute inflammation, chronic inflammation, bleeding disorder, coagulation disorder, and/or liver disease.

The method includes a first step of transducing at least one therapeutic RNA sequence together with at least one therapeutic RNA packaging sequence including an RNA binding protein sequence into progenitors of megakaryocytes and/or CD34-expressing hematopoietic stem and progenitor cells (HSPC), immature megakaryocytes, and/or megakaryocytes, using a DNA-based vector, the therapeutic RNA sequence and/or packaging sequence encoding at least one therapeutic RNA product.

Also, the method comprises a second step of activating the expression of the therapeutic RNA sequence and/or the therapeutic RNA packaging sequence.

Still further, the method comprises a third step of generating platelets from mature megakaryocytes derived from the transduced progenitors of megakaryocytes and/or from the transduced CD34-expressing hematopoietic stem and progenitor cells (HSPCs) and/or from the transduced immature megakaryocytes and/or from the transduced megakaryocytes.

The generated platelet vehicles are functionally reactive to activation with specific agonists.

The generated platelet vehicles are activated by extracellular signals.

The generated platelet vehicles contain the at least one therapeutic RNA product.

The invention is based on the basic idea that megakaryocytes (MKs) (e.g. immortalized MKs), progenitors of megakaryocytes (e.g. immortalized progenitors of megakaryocytes), or CD34-expressing HSPC (e.g. immortalized CD34-expressing HSPC), are transduced to express at least one therapeutic RNA sequence and/or therapeutic RNA packaging sequence at the stage of megakaryocytes, avoiding the alteration of the biology of megakaryocyte progenitor and/or CD34+ HSPC by the expression of the at least one therapeutic RNA sequence and/or the therapeutic RNA packaging sequence. Transduced cells can be then differentiated into mature megakaryocytes, expressing the at least one therapeutic RNA sequence and/or at least one therapeutic RNA packaging sequence, and manipulated to generate functional platelets. The resulting platelets express the respective at least one therapeutic product and can be further used for therapeutic approaches, e.g. for the production of pharmaceutical compositions.

Furthermore, since platelets are functional, they will be activable by extracellular signals. In other words, passive loading of the therapeutic compound inside or at the surface of mature platelets is avoided. Heavy membrane modification is also avoided. Therapeutic RNA is localized within the platelet vesicles, and functional platelets can release their therapeutic RNA cargo upon activation at the targeted site.

The method according to the invention allows for specific loading of therapeutic RNA into megakaryocytes and/or platelet vesicles by expression of therapeutic RNA packaging sequences, allowing microvesicle release at specific target cells and/or tissue after activation from an extracellular signal.

In addition, the risk of therapeutic RNA expression during megakaryopoiesis can be reduced.

The therapeutic RNA platelets vehicle engineered through the method of the invention can allow silencing a gene of interest (e.g., via miRNA, siRNA, shRNA) at a target site, e.g. a cell and/or tissue.

The method is simple and allows the production of engineered therapeutic RNA platelet vehicles on a clinically relevant scale, at comparatively low cost.

After infusion into patients, therapeutic RNA platelet vehicles will circulate in blood in a resting state, tightly controlled by plasmatic and cellular components of blood vessels.

Inside circulating therapeutic RNA platelet vehicles, the at least one therapeutic product encoded by the therapeutic RNA sequence is shielded from inhibition or degradation.

Only after arrest at a specific platelet activation site, such as a bleeding, injury, cancer, or inflammation site, therapeutic RNA platelet vehicles will be activated by extracellular signal, and release their cargo including the at last one therapeutic RNA.

Thus, the therapeutic RNA platelet vehicles produced by the method of the invention can be regarded as pathological process-targeted therapeutic product delivery vehicles.

Preferably, CD34+ HSPCs and/or immortalized MK cell lines and/or MK progenitors are transduced with an expression cassette using lentivirus vector.

The expression cassette can contain either a constitutive promoter (e.g., Spleen focus-forming virus (SFFV) and/or Elongation factor 1a (EF1a)) and/or a megakaryocytic promoter (e.g. GPVI, PF4, CD41), and/or an inducible promoter (e.g. Tetracycline and/or Isopropyl β-D-1-thiogalactopyranoside (IPTG) and/or Cumate-controlled operator system).

Advantageously, the RNA binding protein may be one among HNRNPA2B1, HNRNPC1, RBMX, HNRNPH1, HNRNPK, HNRNPQ, YBOX1, ELAV1, AGO2, IGF2BP1, MEX3C, ANXA2, PDC6I, NUCL, FUS, MVP, LIN28A, SRP14, SRP09, QKI, and TERT.

RNA binding motifs for each of said RNA binding proteins are provided in detail in Table 1.

Advantageously, the extracellular signal may include a protein expressed on the surface of a cell, paracrine signals, and/or autocrine signals.

Advantageously, the therapeutic RNA sequence encodes for at least one of messenger RNA (mRNA), small interfering RNA (siRNA), short hairpin RNA (shRNA), long non-coding RNA (lncRNA), micro RNA (miRNA), or small activating RNA (saRNA).

mRNA is a single-stranded molecule of RNA that corresponds to the genetic sequence of a gene, and is read by a ribosome in the process of synthesizing a protein. mRNA can encode for, e.g., an antibody, a cytokine, a receptor protein, or chimeric receptor protein, or the like.

Thus, RNA material transferred by the therapeutic RNA platelet vehicle can be translated within the target cell as an antibody and/or cytokine for a desired in-situ reaction.

SiRNA is a class of double-stranded RNA at first non-coding RNA molecules, typically 20-24 (normally 21) base pairs in length, similar to miRNA, and operating within the RNA interference (RNAi) pathway. It interferes with the expression of specific genes with complementary nucleotide sequences by degrading mRNA after transcription, preventing translation.

ShRNA is an artificial molecule with a tight hairpin turn that can be used to silence target gene expression via RNA interference.

lncRNA family engulf RNAs longer than 200 nucleotides that are not translated into a functional protein. lncRNA regulate gene expression by direct interaction with DNA, RNA, and/or proteins and modulate chromatin structure and function and the transcription of genes. They can as well affect RNA splicing, stability and translation.

MiRNA is a small single-stranded non-coding RNA molecule (containing about 22 nucleotides) found in plants, animals and some viruses, that functions in RNA silencing and posttranscriptional regulation of gene expression.

MiRNAs function via base-pairing with complementary sequences within mRNA molecules. As a result, these mRNA molecules are silenced, by one or more of the following processes: (1) cleavage of the mRNA strand into two pieces, (2) destabilization of the mRNA through shortening of its poly (A) tail, and (3) less efficient translation of the mRNA into proteins by ribosomes.

saRNA are chemically synthetized 21 nucleotides in length double strand RNA oligonucleotides. They increase expression of their target genes above endogenous levels by binding near or within the target gene promoter.

A specific motif can be added to the therapeutic RNA sequence to ensure binding to a specific therapeutic RNA packaging protein, such as RNA binding protein.

In general, the therapeutic RNA sequence can be single.

In this case, the sequence for the therapeutic compound contains only one therapeutic RNA.

Alternatively, the therapeutic RNA sequence can be tandem for multiplexing. This means that copies of sequences lie adjacent to each other in the same orientation (direct tandem repeats) or in the opposite direction to each other (inverted tandem repeats).

In particular, the therapeutic RNA sequence can be adapted for targeting at least one among breast cancer and/or ovarian cancer, and/or siRNA for BRCA2 mutant.

Additionally or alternatively, the therapeutic RNA sequence can be adapted for inhibition of a member of alt-NHEJ pathway.

Preferably, said member alt-NHEJ pathway is PARP1.

Additionally or alternatively, the therapeutic RNA sequence can be adapted for activation of a member of pro-apoptotic family BH3-only protein by saRNA.

Advantageously, the therapeutic RNA sequence may contain a specific protein binding motif or an EXOmotif comprising GAGAG.

Alternatively, the therapeutic RNA sequence may contain a specific protein binding motif or an EXOmotif comprising GGAG.

Preferably, the specific protein binding motif or EXOmotif has high affinity for a protein selected from the family of RNA-binding protein in exosomes.

Here, the RNA binding protein is preferably the protein counterpart of GAGAG or GGAG, respectively.

Advantageously, the therapeutic RNA and the therapeutic RNA packaging sequence expression can be driven by a megakaryocyte specific promoter.

For instance, said megakaryocyte specific promoter may include GPVI, CD41, PF4, and/or an inducible promoter.

Optional additional activator receptor proteins may be expressed under a constitutive promoter.

Next to the promoter, one or multiple coding sequences for the therapeutic RNA, in particular also referred to as one or multiple therapeutic RNA sequences, can be added. In particular, the expression cassette can be referred to as therapeutic product expression cassette. Also, multiple viruses can be used to express multiple therapeutic products.

Advantageously, the DNA-based vector may further comprise at least one coding sequence for a chimeric or wild type activator receptor protein from the triggering receptor expressed on myeloid cells-like (TREM) family, preferably NCR2 encoding NKp44, TREM1, TREML4 encoding TREM-like 4, TREML2, TREM2, and TREML, and/or from the Notch family, preferably Notch1, Notch2, Notch3, and Notch4, with a wild type or a modified extracellular sequence.

Advantageously, the modified extracellular domain of the chimeric activator receptor protein may consist of or comprise a single-chain variable fragment (scFv) protein, a receptor ligand, a full receptor protein or an extracellular protein domain of a receptor, a nanobody, a designed ankyrin repeat proteins (DARPins), or adapter chimeric antigen receptor (adCAR).

Advantageously, the chimeric activator receptor protein may be constitutively expressed.

The present invention further provides a therapeutic RNA platelet vehicle engineered through the above-described method.

In particular, the therapeutic RNA platelet vehicle so obtained is capable of expressing at least one therapeutic coding sequence and/or at least one coding sequence for a receptor, preferably a cell or tissue or organ specific receptor protein.

Advantageously, said therapeutic RNA platelet vehicle can be used for the treatment, preferably target-specific treatment, of a disease.

For instance, said therapeutic RNA platelet vehicle can be used for the treatment, preferably target-specific treatment, of at least one among cancer, acute inflammation, chronic inflammation, bleeding disorder, coagulation disorder, and/or liver disease.

For instance, said therapeutic RNA platelet vehicle may express at least one therapeutic RNA sequence and/or at least one therapeutic RNA packaging sequence and/or at least one coding for an activator receptor protein, wildtype and/or chimeric, allowing specific targeting such as, e.g., a cell or tissue or organ specific receptor protein.

Platelets naturally express C-type lectin domain family 1 member B (CLEC-2), the receptor for PDPN. Engineered platelets are therefore naturally activated in tissue overexpressing PDPN, such as in PDPN-positive cancer. PDPN overexpression in cancer usually leads to increased metastasis and prothrombotic events.

Thus, immediately after activation, engineered platelets will liberate miRNA and/or siRNA against PDPN in the vicinity of tumors, possibly via microparticles. SiRNA or miRNA will be engulfed or endocytosed by surrounding cells, and their PDPN expression will be downregulated.

Localized downregulation can decrease tumor progression and metastasis.

In healthy tissues, the expression of PDPN does not lead to platelet activation, despite expression of PDPN and CLEC-2.

In particular, this is due to the nature of the vasculature, preventing platelet activation.

Moreover, platelets do not circulate in lymphatic vessels.

However, in PDPN-overexpressing cancer, platelets are activated (supported by literature).

In patients with infused engineered therapeutic RNA platelet vehicles according to the invention, platelets will be resting until being in the tumor vicinity, triggering the release of anti-PDPN products by platelets.

Still further, the present invention provides a pharmaceutical composition comprising therapeutic RNA platelet vehicles engineered through the above-described method.

In particular, said therapeutic RNA platelet vehicles are capable of expressing at least one therapeutic coding sequence and/or at least one coding sequence for a receptor, preferably a cell or tissue or organ specific receptor protein.

Advantageously, said pharmaceutical composition can be used for the treatment, preferably target-specific treatment, of a disease.

For instance, said pharmaceutical composition can be used for the treatment, preferably target-specific treatment, of at least one among cancer, acute inflammation, chronic inflammation, bleeding disorder, coagulation disorder, and/or liver disease.

The pharmaceutical composition may include therapeutic RNA platelet vehicles expressing at least one therapeutic RNA sequence and/or at least one therapeutic RNA packaging sequence and/or at least one coding for an activator receptor protein, wildtype and/or chimeric, allowing specific targeting such as, e.g., a cell or tissue or organ specific receptor protein.

It is important to ensure the sequestration of the therapeutic RNA with the vesicles.

Therefore, packaging proteins are co-expressed with the therapeutic RNA.

Furthermore, the therapeutic RNA is also expressing a specific DNA sequence, called EXOmotif, to specifically bind to the therapeutic RNA packaging proteins.

As a non-limiting example, the therapeutic RNA packaging proteins may consist in or comprise RNA binding proteins with the ability to tether RNA with a specific sequence motif to the vesicles, and localize within the vesicle lumen (Table 1).

Thus, the expression cassette, delivered by a DNA-based vector, can additionally or alternatively) comprise at least one coding sequence for an activator receptor protein (wild type or with a modified extracellular domain), wherein platelets express the activator receptor protein.

In other words, the DNA-based vector can further comprise at least one coding sequence for an activator receptor protein (wild type or with a modified extracellular domain), wherein the platelets express the activator receptor protein.

This allows platelets, expressing the activator receptor protein, to be activated when the ligand of the activator receptor protein is present in the surrounding platelet environment, enabling for precise activation of platelets at a site of interest.

Indeed, for some indications, physiological mechanisms of activation may not be sufficient to: 1) trigger sufficient activation for cargo release, 2) homing at site of interest.

In general, the activator receptor protein can be a non-modified receptor protein (in particular a non-modified cellular receptor protein), a receptor protein complex (in particular a cellular receptor complex), a chimeric receptor protein (in particular a cellular chimeric receptor protein), or a chimeric receptor protein containing a gain-of-function mutation.

The activator receptor protein (wild type or with a modified extracellular domain) is from the triggering receptor expressed on myeloid cells-like (TREM) family and/or from the Notch family with a wild type or modified extracellular sequence.

A receptor complex is composed of at least one core receptor protein and receptor-associated proteins, which have profound effects on the overall receptor structure, function and localization.

A chimeric activator receptor can be defined as a special receptor created in the laboratory that is designed to bind to certain proteins on targeted cells and/or tissues.

CD34+ HSPC, in particular immortalized CD34+ HSPC, or progenitors of megakaryocytes (MKs), in particular immortalized progenitors of megakaryocytes (MKs), or megakaryocytes, in particular immortalized megakaryocytes, can be additionally or alternatively engineered to express at least one activator receptor protein (wild type or with a modified extracellular domain).

Platelets produced form the mature megakaryocytes derived from CD34+ HSPC and/or immortalized CD34+ HSPC, and/or progenitors of megakaryocytes, and/or immortalized progenitors of megakaryocytes and/or megakaryocytes and/or megakaryocytes expressing at least one activator receptor protein (wild type or with a modified extracellular domain), will express the at least one activator receptor protein (wild type or with a modified extracellular domain).

Only after arrest at a specific recruitment site, such as bleeding, injury, cancer, or inflammation, or at a site expressing the ligand of the activator receptor protein (wild type or with a modified extracellular domain) expressed by therapeutic RNA platelet vehicles, platelets will be activated and release their cargo including, e.g., at least one therapeutic compound.

The invention allows for target-specific delivery of at least one therapeutic compound, such as a multispecific drug.

Platelets can be activated by multiple, redundant pathways; therefore, an activator receptor protein (wild type or with a modified extracellular domain) can increase the specificity of the activation, being part of the surface activation matrix.

Overall, only a net intra-platelet signaling in favor of activation will trigger full activation and microparticle release, decreasing the risk of unspecific release of the cargo.

In other words, the method allows for production of platelets expressing a tissue-specific receptor that will facilitate platelet arrest at site of interest, and trigger or potentiate cargo release.

In particular, the coding sequence for the activator receptor protein (wild type or with a modified extracellular domain) can comprise mRNA.

In particular, the activator receptor extracellular domain can be modified and replaced by a single-chain variable fragment (scFv) protein, a receptor ligand, a full receptor protein or an extracellular protein domain of a receptor, a nanobody, a designed ankyrin repeat proteins (DARPins), or adapter chimeric antigen receptor (adCAR).

This can be achieved by expressing a chimeric activator receptor as follows: the extracellular domain contains the tissue specific moiety (e.g. scFv, or DARPin), conjugated to the transmembrane domain and intracellular domain of the triggering receptor expressed on myeloid cells-like (TREM) family and/or from the Notch family. Upon binding to the target of the receptor, endogenous signaling pathway will activate the therapeutic RNA platelet vehicles, allowing precise activation of platelets at a site of interest. To enhance signaling, a gain-of-function mutation can be introduced in the activator receptor protein (wild type or with a modified extracellular domain) coding sequence.

One example of targeting moiety that can be expressed at the surface of engineered platelets is a scFv targeting PDPN, to increase homing of therapeutic platelets to a tumor environment, where CLEC-2 will lead to platelet activation and release of therapeutics.

Another example is to take advantage of the overexpression of transferrin receptor by tumor cells (Gatter et al., 1983).

Therefore, expressing transferrin, or more elegantly a scFv binding to transferrin on engineered platelets, will also improve homing of platelets to the tumor environment. The same strategy can be used by targeting the folate receptor (FR), also expressed in many cancers (Sudimack and Lee, 2000). Finally, all monoclonal antibodies (mAb) targeting cancer cells, such as EGFR (e.g. panitumumab), could be used by generating scFv from the Fab domain of the mAbs, using current molecular biology methods.

Another example is the expression of an adCAR on platelets. An adCAR is a chimeric receptor binding specifically to another targeting protein (or targeting module), such as a monoclonal Antibody (mAb).

A therapeutic mAb is conjugated to a biotin or another epitope tag (to form a linker-label epitope (LLE). Therapeutic mAb is dosed or infused according to its pharmacokinetic and distribution parameters. At different time points during treatment, engineered platelets expressing an adCAR will be infused. Platelets will then have a specific homing behavior, towards tissues expressing the target of the mAb. Upon arrest and full activation, platelet will release their therapeutic cargo.

Alternatively, the coding sequence could encode for a radio-sensitizer, to increase sensitivity of platelet-targeted tissue for radiotherapy. Indeed, some miRNA such as miR-621, targeting SETDB1 in hepatocellular carcinoma, can act as radiosensitizer (Shao et al., 2019). Similarly, a short siRNA against S100A4 enhances the radiosensitivity of human adenocarcinome epithelial cells (Qi, Qiao and Zhuang, 2016).

In general, the RNA platelet vehicles are produced for therapeutic use.

For instance, RNA platelet vehicles can be used for the preparation of a pharmaceutical composition, as mentioned in the foregoing.

If needed, therapeutic RNA platelet vehicles can express an activator receptor (wild type or with a modified extracellular domain), for use for more sensitive targets such as a cell or tissue or organ specific receptor protein, for the treatment of a disease.

For instance, a pharmaceutical composition may comprise therapeutic RNA platelet vehicles expressing siRNA and/or miRNA and/or shRNA and/or saRNA and/or lncRNA against a defined protein and/or group of proteins.

After administration of the pharmaceutical composition, therapeutic RNA platelet vehicles will be activated in a tissue by extracellular signal within the microenvironment of the therapeutic RNA platelet vehicle recruitment site. Immediately after activation, platelets may liberate siRNA and/or miRNA and/or shRNA and/or saRNA and/or lncRNA in the vicinity by microparticles (exosomes and ectosomes). These microparticles may be engulfed by surrounding cells, and the defined protein and/or group of proteins expression could be deregulated. Localized deregulation of a defined protein or a group of proteins, for instance in breast or ovarian cancer multiplexing an siRNA for BRCA2 mutant together with inhibition of a member of alt-NHEJ pathway, e.g. PARP1, while activating a member of pro-apoptotic family BH3-only protein by saRNA, could decrease tumor progression and metastasis.

Additionally or alternatively, the pharmaceutical preparation may comprise therapeutic RNA platelet vehicles expressing at least one coding sequence for an activator receptor protein.

For instance, the protein can be from the TREM or the Notch family.

The pharmaceutical composition can be used in a method for the treatment of at least one of cancer, acute inflammation, chronic inflammation, bleeding disorder, coagulation disorder, and/or liver disease.

It is known that platelets are physiologically activated in these disease conditions.

### BRIEF DESCRIPTIONS OF THE DRAWINGS

Further details and advantages of the present invention will now be disclosed in connection with the drawings.

It is shown in:

FIG. 1 a block diagram schematically illustrating a method for producing therapeutic RNA platelet vehicles, according to an embodiment of the present invention;

FIG. 2 a schematic illustration of how genetic modification of an immortalized megakaryocyte leads to the production engineered therapeutic RNA platelet vehicles, in the method of FIG. 1;

FIG. 3 a schematic illustration of the release of microparticles from therapeutic RNA platelet vehicles to a target cell;

FIG. 4 a schematic view of engineered therapeutic RNA platelet vehicles releasing a therapeutic compound at a target site of interest;

FIG. 5 an example of a lentiviral transfer plasmid with an expression cassette comprising a therapeutic coding sequence;

FIGS. 6a-b an example of protein expression driven by a megakaryocytic promoter;

FIGS. 7a-c an example of a miRNA sequence, introduced into megakaryocyte progenitor, targeting B2M;

FIGS. 8a-b an example of a chimeric receptor expressed on the surface of megakaryocyte progenitors and mature megakaryocytes; and

FIGS. 9a-c an example of shRNA expressed in platelets and megakaryocytes after transduction of CD34+ cells.

### DETAILED DESCRIPTION OF THE INVENTION

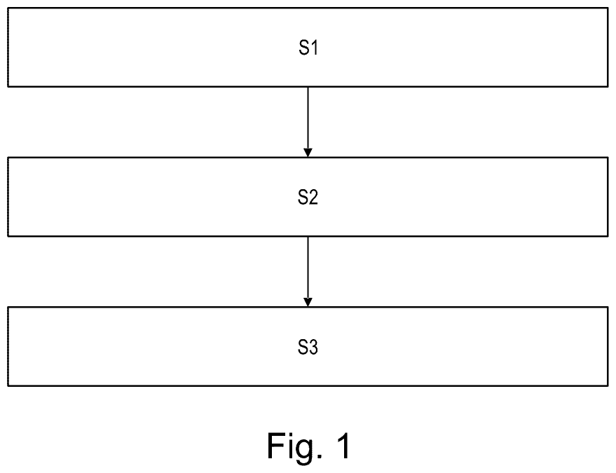

FIG. 1 shows a block diagram illustrating a method for producing engineered therapeutic RNA platelet vehicles, according to an embodiment of the present invention.

The method includes at least three steps, referred to as S1 to S3 (FIG. 1).

The first step S1 comprises transducing at least one therapeutic RNA sequence together with at least one therapeutic RNA packaging sequence including an RNA binding protein sequence into progenitors of megakaryocytes and/or CD34-expressing hematopoietic stem and progenitor cells (HSPC), immature megakaryocytes, and/or megakaryocytes, using a DNA-based vector, the therapeutic RNA sequence and packaging sequence encoding at least one therapeutic RNA product.

The second step S2 comprises activating the expression of the therapeutic RNA sequence and/or the therapeutic RNA packaging sequence.

The third step S3 comprises generating platelets from mature megakaryocytes derived from the transduced progenitors of megakaryocytes and/or from the transduced CD34-expressing hematopoietic stem and progenitor cells and/or from the transduced immature megakaryocytes and/or from the transduced megakaryocytes.

The expression activation step is regulated to occur during maturation towards megakaryocytes and/or platelet formation.

The generated platelet vehicles are functionally reactive to activation with specific agonists.

The generated platelet vehicles are activated by extracellular signals.

The generated platelet vehicles contain the at least one therapeutic RNA product.

In the present embodiment, the RNA binding protein is one among HNRNPA2B1, HNRNPC1, RBMX, HNRNPH1, HNRNPK, HNRNPQ, YBOX1, ELAV1, AGO2, IGF2BP1, MEX3C, ANXA2, PDC6I, NUCL, FUS, MVP, LIN28A, SRP14, SRP09, QKI, and TERT (Table 1).

In the present embodiment, the extracellular signal includes a protein expressed on the surface of a cell, paracrine signals, and/or autocrine signals.

In the present embodiment, the therapeutic RNA sequence encodes for at least one of messenger RNA (mRNA), small interfering RNA (siRNA), short hairpin RNA (shRNA), long non-coding RNA (lncRNA), micro RNA (miRNA), or small activating RNA (saRNA).

In general, the therapeutic RNA sequence can be single.

In this case, the sequence for a therapeutic compound contains only one therapeutic RNA.

Alternatively, the therapeutic RNA sequence can be tandem for multiplexing.

This means that copies of sequences lie adjacent to each other in the same orientation (direct tandem repeats) or in the opposite direction to each other (inverted tandem repeats).

The therapeutic RNA sequence can be adapted for targeting at least one among breast cancer and/or ovarian cancer, and/or siRNA for BRCA2 mutant.

Additionally or alternatively, the therapeutic RNA sequence can be adapted for inhibition of a member of alt-NHEJ pathway.

Preferably, said member alt-NHEJ pathway is PARP1.

Additionally or alternatively, the therapeutic RNA sequence can be adapted for activation of a member of pro-apoptotic family BH3-only protein by saRNA.

In the present embodiment, the therapeutic RNA sequence contains a specific protein binding motif or an EXOmotif comprising GAGAG or GGAG

In the present embodiment, the RNA binding protein is the protein counterpart of GAGAG or GGAG.

In the present embodiment, the therapeutic RNA and the therapeutic RNA packaging sequence expression is driven by a megakaryocyte specific promoter.

In particular, in the present embodiment, said megakaryocyte specific promoter preferably includes GPVI, CD41, PF4, and/or an inducible promoter.

In the present embodiment, the DNA-based vector further comprises at least one coding sequence for a chimeric or wild type activator receptor protein from the triggering receptor expressed on myeloid cells-like (TREM) family, preferably NCR2 encoding NKp44, TREM1, TREML4 encoding TREM-like 4, TREML2, TREM2, and TREML1, and/or from the Notch family, preferably Notch1, Notch2, Notch3, and Notch4, with a wild type or a modified extracellular sequence.

In the present embodiment, the modified extracellular domain of the chimeric activator receptor protein consists of or comprises one of a single-chain variable fragment (scFv) protein, a receptor ligand, a full receptor protein or an extracellular protein domain of a receptor, a nanobody, a designed ankyrin repeat proteins (DARPins), or adapter chimeric antigen receptor (adCAR).

In the present embodiment, the chimeric activator receptor protein is constitutively expressed.

A lentivirus can be used to transduce an expression cassette into cells. E.g. human cells.

In particular, the expression cassette may comprise a promoter. Next to the promoter, a coding sequence for the therapeutic compound is added.

Alternatively, the expression cassette can be transduced into immature megakaryocytes.

Alternatively, mature megakaryocytes or any progenitors of megakaryocytes can be transduced.

For instance, the expression cassette may comprise a therapeutic RNA sequence.

Alternatively, the expression cassette may comprise more than one therapeutic RNA sequence.

The therapeutic RNA platelet vehicles express at least one therapeutic product.

In other words, the therapeutic RNA platelet vehicles contain the at least one therapeutic product.

Not shown here is that the DNA-based vector can further comprise at least one coding sequence for a receptor protein, e.g. a cell or tissue or organ specific receptor, wherein the platelets express the receptor protein.

Also not shown is that the receptor protein can be a receptor complex protein.

Alternatively, the receptor protein can be a non-modified receptor protein or a chimeric receptor protein, or a chimeric receptor protein containing a gain-of-function mutation.

Also not shown here is that the coding sequence for the receptor protein can comprise mRNA.

The receptor can be specific for a cell type, a tissue or an organ.

Also not shown is that the at least one coding sequence for the receptor protein can encode for a scFv.

Also not shown is that the coding sequence also can encode for a scFv together with a signaling intracellular domain.

Alternatively, the coding sequence can only encode for a single-chain variable fragment (scFv), a ligand, a receptor, a nanobody, designed ankyrin repeat proteins (DARPins), a chimeric antigen receptor, and/or an adapter chimeric antigen receptor (adCAR), in particular together with a transmembrane domain or anchoring region such as a GPI-anchor.

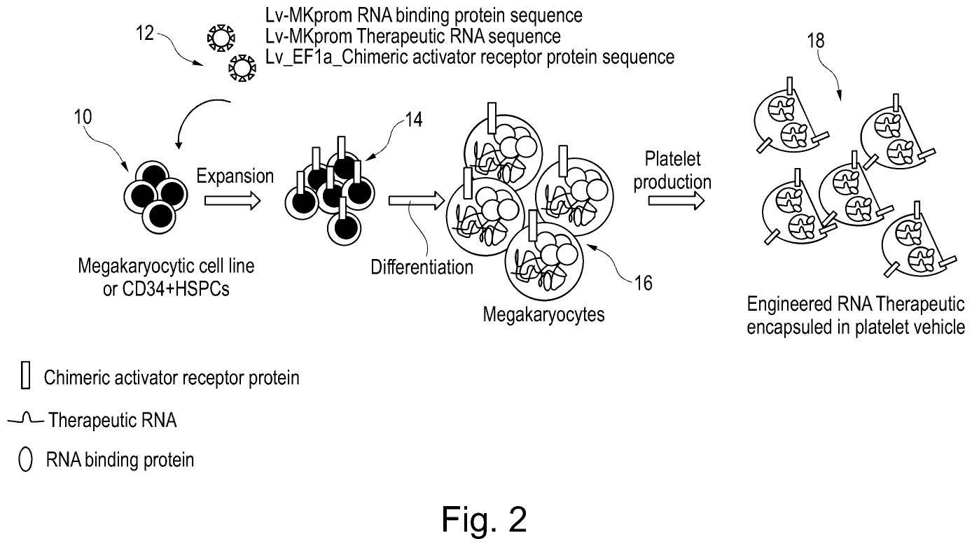

FIG. 2 provides a schematic illustration of how genetic modification of an immortalized megakaryocyte leads to the production engineered therapeutic RNA platelet vehicles, in the method of the invention.

Megakaryocytic progenitor cells 10 are first transduced with lentiviral particles 12 to express therapeutic RNA compounds.

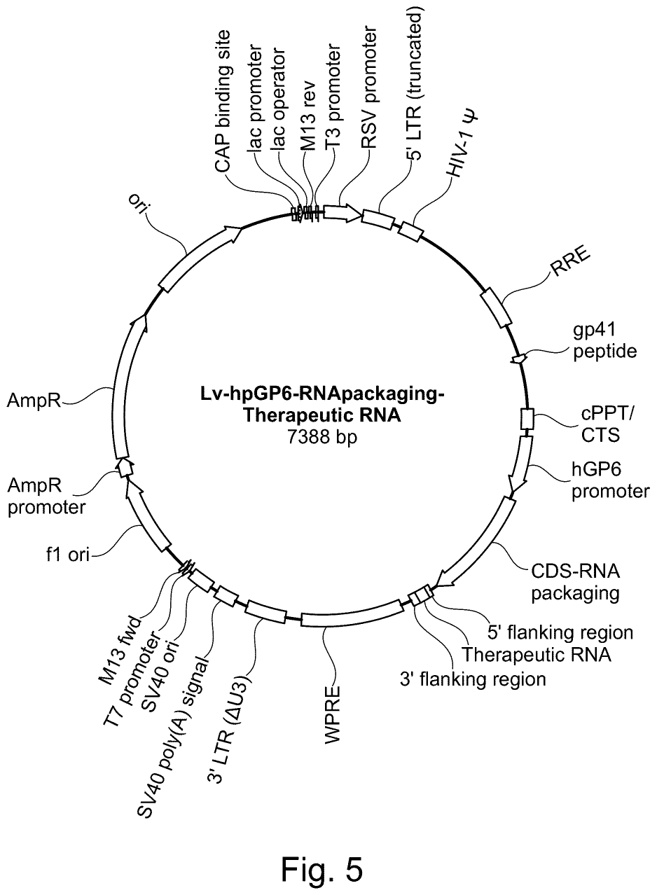

An example of a lentiviral particle, i.e. a lentiviral transfer plasmid, is illustrated in FIG. 5.

In particular, here, the lentiviral particles 12 transduce an expression cassette comprising a therapeutic sequence.

Successfully transduced cells 14 are then selected.

Cells 10 are expanded in a selective medium and finally differentiated into mature megakaryocytes 16.

Upon maturation, megakaryocytes express the therapeutic RNA sequence and/or the packaging protein, here an RNA binding protein. NA binding proteins allow for the localization of the therapeutic RNA sequence within vesicles.

After their transfer into a microfluidic device to generate adhesion and shear, mature megakaryocytes 16 are releasing platelets 18 containing the exogenous RNA therapeutic compound, encoded by the therapeutic sequence.

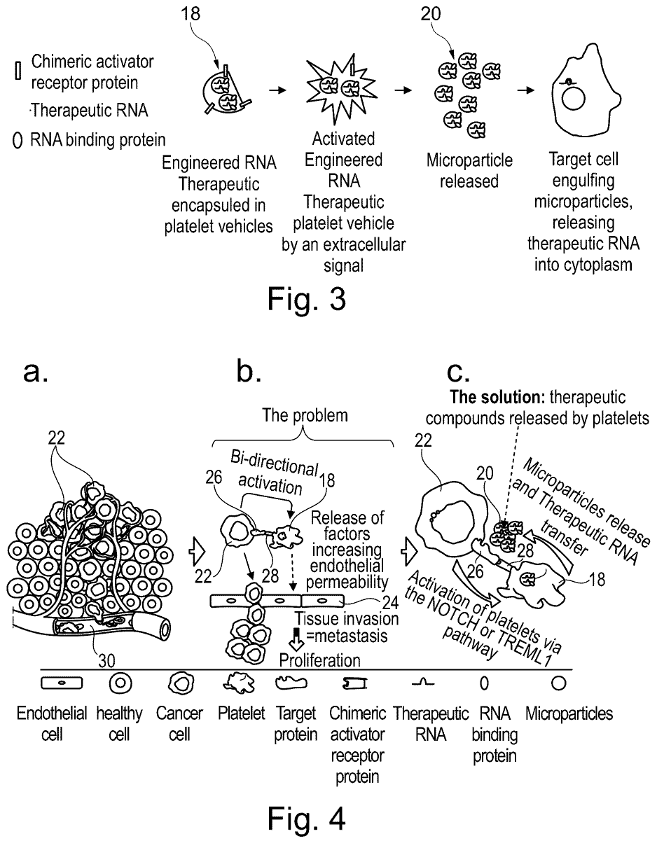

FIG. 3 illustrates the release of microparticles from platelets.

In particular, shown are platelets 18.

In disease tissue, such as cancer, liver disease, or during bleeding, platelets 18 are activated by environmental factors, such as cytokines, thrombin and/or collagen, and release the content of their granules, as well as microparticles 20.

Platelets 18 produced by the method according to the present invention (FIGS. 1-2) express at least one therapeutic sequence and thus microparticles 20, released from these platelets 18, comprise the respective therapeutic RNA product.

FIG. 4 shows a schematic view of engineered platelets releasing a therapeutic compound at a target site of interest.

Cancer cells 22 in tumors release metastatic cells 22 in the blood stream 30. Further, cancer cells 22 or metastatic cells 22 overexpress a specific protein or group of proteins 26.

In platelets 18 expressing a chimeric activator receptor, such as NOTCH1 or TREML1 28, the wild type or the modified extracellular domain is a receptor for the tumoral protein 26. Here, platelets 18 are engineered to also express a therapeutic RNA product against a specific oncogene.

Platelets 18, here wild-type platelets 18, are activated by metastatic cancer cells 22 in the blood stream 30, by the PDPN-CLEC-2 pathway, releasing microparticles 20 comprising chemokines, cytokines, and factors leading to increased endothelial cell 24 permeability, invasion of cancer cells 22 into tissue, and proliferation. Engineered platelets 18, activated by extracellular signal, also liberate miRNA (alternatively or additionally siRNA) against a specific protein or group of proteins in the vicinity of tumors, via microparticles 20.

These microparticles 20 will be engulfed by surrounding cancer or metastatic cells 22, and a specific protein or group of proteins will be deregulated.

A specific protein or group of proteins deregulation can decrease tumor progression and/or metastasis.

In other words, engineered platelets 18, activated by extracellular signal pathway, are capable of releasing exogenous therapeutics, interfering with disease progression. This generates a virtuous circle, with less platelet 18 activation and decreased tumor growth.

FIG. 5 shows an example of a lentiviral transfer plasmid with an expression cassette comprising a therapeutic coding sequence.

Of importance, the plasmid is showing the GP6 promotor allowing for expression specifically in megakaryocytes.

The transfer plasmid further comprises the following segments/sequences:

cPPT/CTS segment (short for central polypurine tract/chain termination sequence): this sequence helps to increase the efficiency of transduction.

Here, the cPPT/CTS segment comprises 118 base pairs.

RRE (short for Rev response element, in particular HIV-1 Rev response element): this allows the nuclear export of viral RNA by the viral Rev protein during viral packaging. In particular, all lentiviruses encode a regulatory protein Rev that is essential for posttranscriptional transport of the unspliced and incompletely spliced viral mRNAs from nuclei to cytoplasm. The Rev protein acts via binding to an RNA structural element known as the Rev responsive element (RRE).

HIV-1φ: HIV-1 packaging signal required for the packaging of viral RNA into virus.

3′LTR (ΔU3) (short for 3′ long terminal repeat that deletes the U3 region): a truncated version of the HIV-1 3′ long terminal repeat that deletes the U3 region. This leads to the self-inactivation of the promoter activity of the 5′ LTR upon viral vector integration into the host genome (due to the fact that 3′ LTR is copied onto 5′ LTR during viral integration). The polyadenylation signal contained in 3′ LTR-ΔU3 serves to terminate all upstream transcripts produced both during viral packaging and after viral integration into the host genome.

5′ LTR (short for 5′ long terminal repeat): a deleted version of the HIV-1 5′ long terminal repeat. In wildtype lentivirus, 5′ LTR and 3′ LTR are essentially identical in sequence. They reside on two ends of the viral genome and point in the same direction. Upon viral integration, the 3′ LTR sequence is copied onto the 5′ LTR. The LTRs carry both promoter and polyadenylation function, such that in wildtype virus, the 5′ LTR acts as a promoter to drive the transcription of the viral genome, while the 3′ LTR acts as a polyadenylation signal to terminate the upstream transcript. On the present vector, 5′ LTR-ΔU3 is deleted for a region that is required for the LTR's promoter activity normally facilitated by the viral transcription factor Tat. This does not affect the production of viral RNA during packaging because the promoter function is supplemented by the CMV promoter engineered upstream of Δ5′ LTR.

RSV promoter/enhancer (short for Rous sarcoma virus enhancer/promoter): enables production of viral RNA during packaging to compensate for the partially deleted 5′ LTR.

AmpR (short for ampicillin resistance): nucleotide sequence for the ampicillin resistance gene. It allows the plasmid to be maintained by ampicillin selection in E. coli.

Ori (short for origin of replication): particular sequence in the vector at which replication is initiated.

SV40 ori (short for Simian Virus 40 origin for replication): particular sequence in the vector at which replication is initiated bidirectionally.

WPRE (short for Woodchuck Hepatitis Virus Posttranscriptional Regulatory Element): it enhances viral RNA stability in packaging cells, leading to higher titer of packaged virus.

3′ flanking region: a region of DNA that is adjacent to the 3′ end of the therapeutic coding sequence, allows formation of functional pre-miRNA.

5′ flanking region: a region of DNA that is adjacent to the 5′ end of the therapeutic coding sequence, allows formation of functional pre-miRNA.

An exemplary therapeutic RNA sequence can be miR, representing a sequence for expressing miRNA; therapeutic sequence of the pre-miRNA (could alternatively be, e.g., sequence for expressing siRNA).

Coding human sequence (5′-3′, stemloop-forming precursor RNAs) for miR-29b is:

SEQ ID NO. 3:
CUUCAGGAAGCUGGUUUCAUAUGGUGGUUUAGAUUUAAAUAGUGAUU
GUCUAGCACCAUUUGAAAUCAGUGUUCUUGGGGG
(source: www.mirbase.org).

Coding human sequence (5′-3′, stemloop-forming precursor RNAs) for miR-125a is:

SEQ ID NO. 4:
UGCCAGUCUCUAGGUCCCUGAGACCCUUUAACCUGUGAGGACAUCCAG
GGUCACAGGUGAGGUUCUUGGGAGCCUGGCGUCUGGCC
(source: www.mirbase.org).

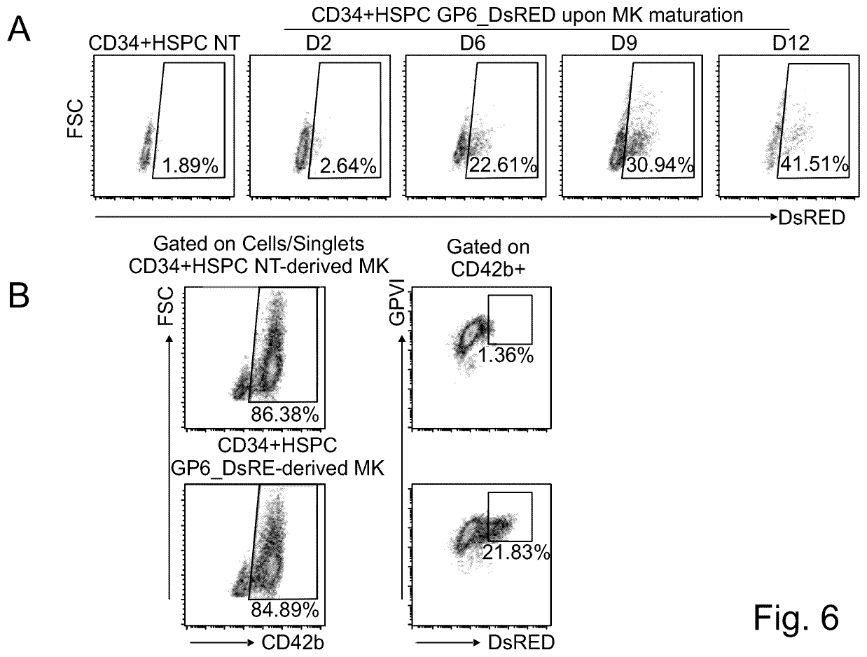

FIG. 6 is a diagram showing that it is possible to control the expression of proteins and ensure the expression only during the maturation towards megakaryocytes.

CD34+ HSPCs were transduced using lentiviral particles to express DsRED fluorescent protein under the control of a megakaryocyte promoter, here a GPVI promoter.

Cells were then differentiated towards megakaryocytes.

FIG. 6A shows an increase of DsRED positive cell within the differentiation course.

FIG. 6B confirms the expression of the DsRED only when GPVI protein is expressed in transduced cells.

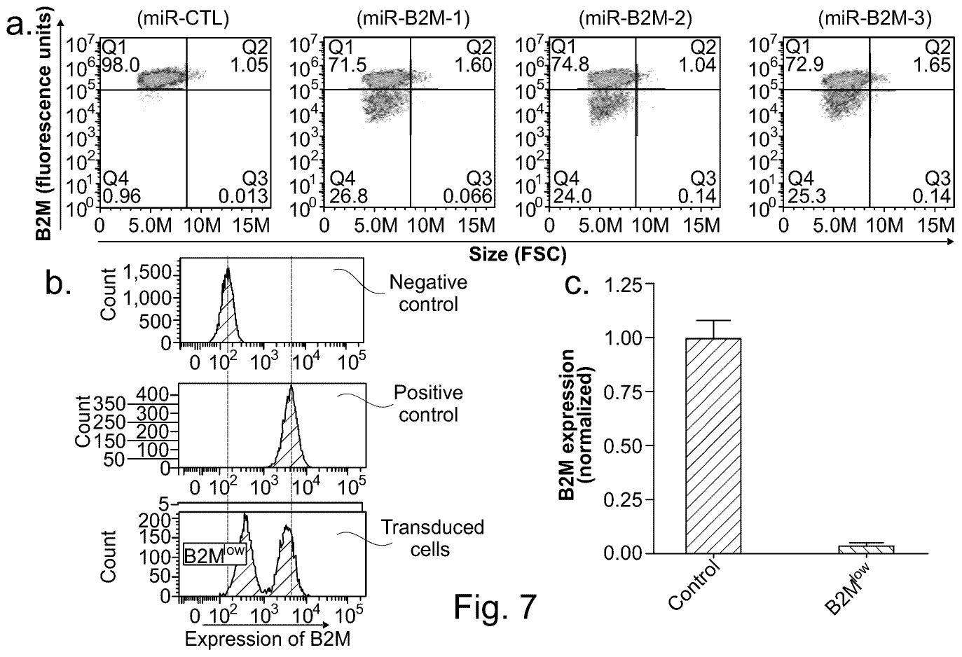

FIG. 7, in particular as a prove of principle, shows expression and biological activity of a transgenic miRNA sequence in megakaryocytes.

Three miRNA sequences targeting human beta-2-microglobulin (B2M), namely:

miR-B2M-1, SEQ ID NO. 5:
GAAUCUUUGGAGUACGCUGGAUGUUUUGGCCACUGACUGACAUCCAGC
GCUCCAAAGAUU;
miR-B2M-2, SEQ ID NO. 6:
GAAACCUGAAUCUGGAGUACGUUUUGGCCACUGACUGACGUACUCCAG
AUUCAGGUUU;
miR-B2M-3, SEQ ID NO. 7:
GAGUAAGUCAACUUCAAUGUCGGUUUUGGCCACUGACUGACCGACAUU
GGUUGACUUACU,
and
one non-targeting control sequence, i.e. miR-
CTL, SEQ ID NO. 8:
GAAAUGUACUGCGCGUGGAGACGUUUUGGCCACUGACUGACGUCUCCA
CGCAGUACAUUU,

were introduced, in separate experiments, into megakaryocyte progenitors using third generation lentiviral vectors, under the control of a spleen focus-forming virus promoter (pSFFV).

After maturation into megakaryocytes, using a cell culture media based on Iscove's Modified Dulbecco's Medium (IMDM), supplemented with stem cell factor (SCF), interleukin-3 (IL-3) and thrombopoietin (TPO) receptor agonist peptide (Cwirla et al., 1997), expression of B2M has been assessed by flow cytometry (cf. FIG. 7a).

All three specific miRNA sequences (i.e. miR-B2M-1, miR-B2M-2, miR-B2M-3) decreased the expression of B2M.

MiR-B2M-1 was chosen for further analysis.

In a next step, MEG-01, a megakaryocyte cell line, was transduced with lentiviral vectors containing miR-B2M-1. After cell sorting to select a population expressing a low level of B2M (defined as B2Mlow, cf. FIG. 7b, residual expression of 6% compared to control, measured by flow cytometry, using antibody clone 2M2 (official name beta-2-microglobulin, also known as IMD43) (from BioLegend), low level of B2M expression was confirmed (cf. FIG. 7c), a residual expression of 4% compared to control (normalized to Hypoxanthine Phosphoribosyltransferase 1 (HPRT1) expression), by real time qPCR, using primers and internal probes from Integrated DNA Technologies (B2M: Hs.PT.58v.18759587; HPRT1: Hs.PT.58v.4562157).

In a next step, platelets can be generated from the transduced megakaryocytes, the platelets showing low expression of B2M.

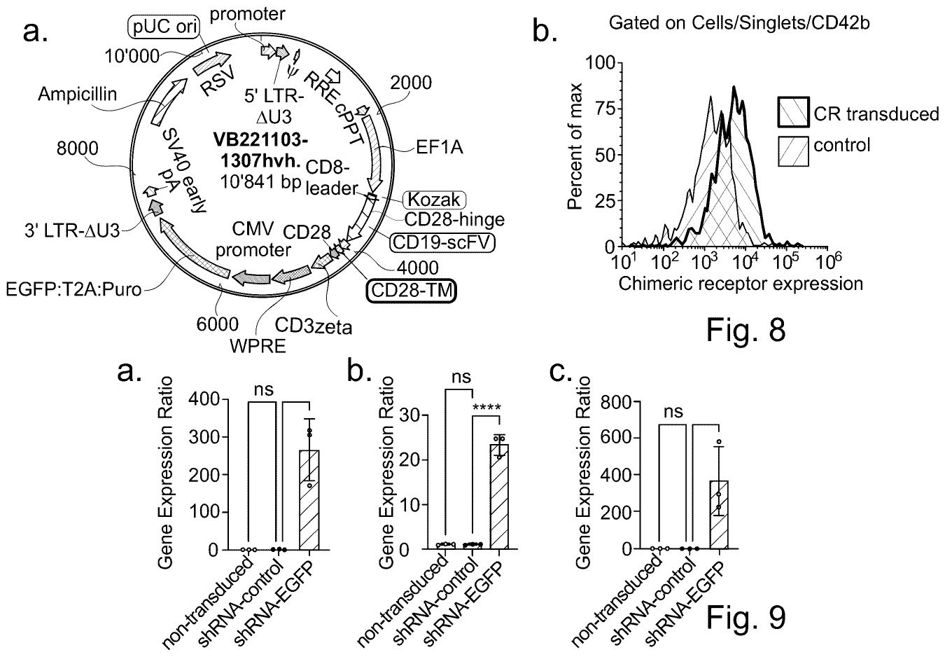

FIG. 8a shows an example of forced expression of chimeric receptor at the surface of megakaryocytes.

Human CD34+ cells have been transduced with a lentiviral vector to express a chimeric antigen receptor (CAR) protein (CD19-scFv/CD28/CD3zeta, expression plasmid for the lentiviral vector shown in FIG. 8a).

After 12 days of differentiation towards megakaryocytes in a cell culture media containing TPO receptor agonists peptides, an expression of the CAR at the surface of the megakaryocytes (CD42b⁺) was observed by flow cytometry, using a fusion protein containing the human CD19 extracellular domain and a mutated human IgG1 Fc region (biotinylated CD19-CAR detection Reagent, Miltenyi Biotec), followed by detection with an anti-biotin antibody, labelled with APC-Vio770.

FIG. 8b shows the expression of a CAR capable to bind CD19.

In a next step, platelets can be generated from the megakaryocytes derived from the transduced CD34⁺ cells, the platelets showing expression of a CAR capable to bind to CD19, confirming that platelets express surface chimeric receptor introduced in megakaryocytes.

FIG. 9 shows an example of shRNA expressed in platelets and megakaryocytes after transduction of CD34⁺ cells. Human CD34⁺ cells have been transduced with a lentiviral vector to express a shRNA targeting enhanced green fluorescent protein (EGFP) (shRNA-EGFP).

By RT-qPCR and using stem-loop RT primers (Chen et al., 2005), expression of shRNA-EGFP was quantified in CD34⁺ cells (starting cells, cf. FIG. 9a), in CD34⁺-derived megakaryocytes (FIG. 9b), and in megakaryocyte-derived platelets (FIG. 9c).

ShRNA-EGFP was clearly detected in all cells and in platelets. Statistics: * P<0.05, ** P<0.01, P<0.001, **** P<0.0001, one-way ANOVA with Sidak correction.

The present invention further provides a therapeutic RNA platelet vehicle engineered through the above-described method (FIGS. 1-2), said therapeutic RNA platelet vehicle expressing at least one therapeutic coding sequence and/or at least one coding sequence for a receptor, preferably a cell or tissue or organ specific receptor protein.

In particular, the therapeutic RNA platelet vehicle so obtained can be used for the treatment of a disease, in particular target-specific treatment of a disease.

For instance, the therapeutic RNA platelet vehicle so obtained can be used for the treatment, in particular target-specific treatment, of at least one among cancer, acute inflammation, chronic inflammation, bleeding disorder, coagulation disorder, and/or liver disease.

Still further, the present invention provides a pharmaceutical composition comprising therapeutic RNA platelet vehicles engineered through the above-described method (FIGS. 1-2), said therapeutic RNA platelet vehicles expressing at least one therapeutic sequence and/or at least one coding sequence for a receptor, preferably a cell or tissue or organ specific receptor protein.

In particular, the pharmaceutical composition can be used for the treatment of a disease, in particular target-specific treatment of a disease.

For instance, the pharmaceutical composition can be used for the treatment, in particular target-specific treatment, of at least one among cancer, acute inflammation, chronic inflammation, bleeding disorder, coagulation disorder, and/or liver disease.

TABLE 1
RNA binding protein identified with extracellular vesicles and their
respective RNA binding motif (Reviewed in Fabbiano et al., 2020)

| | Uniprot | |
|---|---|---|
| Protein | Entry ID | RNA binding motifs |
| HNRNPA2B1 | P22626 | A/G-rich motifs, 5′-AGG, 5′-UAG, 5′-GGAG |
| HNRNPC1 | P07910 | AU-rich elements |
| RBMX | P38159 | 5′-CC[A/C]-rich |
| HNRNPH1 | P31943 | 5′-GGGA |
| HNRNPK | P61978 | 5′-UC₃₋₄(U/A)₂ |
| HNRNPQ | O60506 | 5′-AYAAYY, 5′-UAUYRR, 5′-GGCU |
| YBOX1 | P67809 | 5′-ACCAGCCU, 5′-CAGUGAGC, 5′-UAAUCCCA |
| ELAV1 | Q15717 | AU-rich elements |
| AGO2 | Q9UKV8 | G-rich sequences, 5′-GCACUU |
| IGF2BP1 | Q9NZI8 | 5′-GGACU, 5′-ACACCC, 5′-AAGCACCCGUU |
| | | (SEQ ID NO. 9), 5′-GG(m6A)C |
| MEX3C | Q5U5Q3 | 5′-CAGAGUUUAG |
| ANXA2 | P07355 | 5′-AA(C/G)(A/U)G |
| PDC6I | Q8WUM4 | |
| NUCL | P19338 | 5′-UUAGGG, 5′-UCCCGA |
| FUS | P35637 | |
| MVP | Q14764 | Aspecific, RNA-mediated |
| LIN28A | Q9H9Z2 | 5′-GGAGG/GGAGA |
| SRP14 | P37108 | |
| SRP09 | P49458 | |
| QKI | Q96PU8 | 5′-NACUAAY-N(1-20)-UAAY (SEQ ID NO. 10) |
| TERT | O14746 | |

### BIBLIOGRAPHY

Barrow, A. D. et al. (2004) ‘Cutting Edge: TREM-Like Transcript-1, a Platelet Immunoreceptor Tyrosine-Based Inhibition Motif Encoding Costimulatory Immunoreceptor that Enhances, Rather than Inhibits, Calcium Signaling via SHP-2 1’, The Journal of Immunology, 172 (10), pp. 5838-5842. Available at: doi.org/10.4049/jimmunol.172.10.5838.
Burnier, L. et al. (2009) ‘Cell-derived microparticles in haemostasis and vascular medicine’, Thrombosis and Haemostasis, 101 (3), pp. 439-451.
Chaurasia, S. N. et al. (2022) ‘Notch signaling functions in noncanonical juxtacrine manner in platelets to amplify thrombogenicity’, eLife. Edited by R. Baiocchi and M. Zaidi, 11, p. e79590. Available at: doi.org/10.7554/eLife.79590.
Chen, C. et al. (2005) ‘Real-time quantification of microRNAs by stem-loop RT-PCR’, Nucleic Acids Research, 33 (20), p. e179. Available at: doi.org/10.1093/nar/gni178.
Coxon, C. H., Geer, M. J. and Senis, Y. A. (2017) ‘ITIM receptors: more than just inhibitors of platelet activation’, Blood, 129 (26), pp. 3407-3418. Available at: doi.org/10.1182/blood-2016-12-720185.
Cwirla, S. E. et al. (1997) ‘Peptide agonist of the thrombopoietin receptor as potent as the natural cytokine’, Science (New York, N.Y.), 276 (5319), pp. 1696-1699. Available at: doi.org/10.1126/science.276.5319.1696.
Dunton, T. A. et al. (2014) ‘The Free Energy Landscape of Dimerization of a Membrane Protein, NanC’, PLOS Computational Biology, 10 (1), p. e1003417. Available at: doi.org/10.1371/journal.pcbi.1003417.
Fabbiano, F. et al. (2020) ‘RNA packaging into extracellular vesicles: An orchestra of RNA-binding proteins?’, Journal of Extracellular Vesicles, 10 (2), p. e12043. Available at: doi.org/10.1002/jev2.12043.
Fowler, P. W. et al. (2016) ‘Membrane stiffness is modified by integral membrane proteins’, Soft Matter, 12 (37), pp. 7792-7803. Available at: doi.org/10.1039/C6SM01186A.
Gatter, K. C. et al. (1983) ‘Transferrin receptors in human tissues: their distribution and possible clinical relevance’, Journal of Clinical Pathology, 36 (5), pp. 539-545. Available at: doi.org/10.1136/jcp.36.5.539.
GPP Web Portal—Clone Details (no date). Available at: portals.broadinstitute.org/gpp/public/clone/details?cloneId=TRCN0000231756 (Accessed: 11 Mar. 2024).
Meyer, F. J.-M. de, Venturoli, M. and Smit, B. (2008) ‘Molecular Simulations of Lipid-Mediated Protein-Protein Interactions’, Biophysical Journal, 95 (4), pp. 1851-1865. Available at: doi.org/10.1529/biophysj.107.124164.
Parton, D. L., Klingelhoefer, J. W. and Sansom, M.S.P. (2011) ‘Aggregation of Model Membrane Proteins, Modulated by Hydrophobic Mismatch, Membrane Curvature, and Protein Class’, Biophysical Journal, 101 (3), pp. 691-699. Available at: doi.org/10.1016/j.bpj.2011.06.048.
Qi, R., Qiao, T. and Zhuang, X. (2016) ‘Small interfering RNA targeting S100A4 sensitizes non-small-cell lung cancer cells (A549) to radiation treatment’, OncoTargets and Therapy, 9, pp. 3753-3762. Available at: doi.org/10.2147/OTT.S106557.
Shao, Y. et al. (2019) ‘MicroRNA-621 Acts as a Tumor Radiosensitizer by Directly Targeting SETDB1 in Hepatocellular Carcinoma’, Molecular Therapy: The Journal of the American Society of Gene Therapy, 27 (2), pp. 355-364. Available at: doi.org/10.1016/j.ymthe.2018.11.005.
Smith, C. W. et al. (2018) ‘TREM-like transcript 1: a more sensitive marker of platelet activation than P-selectin in humans and mice’, Blood Advances, 2 (16), pp. 2072-2078. Available at: doi.org/10.1182/bloodadvances.2018017756.
Sudimack, J. and Lee, R. J. (2000) ‘Targeted drug delivery via the folate receptor’, Advanced Drug Delivery Reviews, 41 (2), pp. 147-162. Available at: doi.org/10.1016/s0169-409x (99) 00062-9.
van Uitert, I., Le Gac, S. and van den Berg, A. (2010) ‘The influence of different membrane components on the electrical stability of bilayer lipid membranes’, Biochimica et Biophysica Acta (BBA)—Biomembranes, 1798 (1), pp. 21-31. Available at: doi.org/10.1016/j.bbamem.2009.10.003.
Washington, A. V. et al. (2004) ‘A TREM family member, TLT-1, is found exclusively in the a-granules of megakaryocytes and platelets’, Blood, 104 (4), pp. 1042-1047. Available at: doi.org/10.1182/blood-20Apr. 1, 0315.
Washington, A. V. et al. (2009) ‘TREM-like transcript-1 protects against inflammation-associated hemorrhage by facilitating platelet aggregation in mice and humans’, The Journal of Clinical Investigation, 119 (6), pp. 1489-1501. Available at: doi.org/10.1172/JCI36175.
Washington, A. V., Quigley, L. and Mc Vicar, D. W. (2002) ‘Initial characterization of TREM-like transcript (TLT)-1: a putative inhibitory receptor within the TREM cluster’, Blood, 100 (10), pp. 3822-3824. Available at: doi.org/10.1182/blood-20Feb. 2, 0523.

### REFERENCES

10 megakaryocytic progenitor cells (megakaryocytic cell line)
12 lentiviral particles (vector)
14 clones, successfully transduced cells
16 mature megakaryocytes
18 platelet, engineered platelet
20 microparticles
22 cancer cell, metastatic cell
24 endothelial cell
26 tumoral protein
28 chimeric activator receptor (e.g. NOTCH1 or TREML1)
30 bloodstream
S1 method step 1
S2 method step 2
S3 method step 3

1. A method for producing therapeutic RNA platelet vehicles, the method comprising at least the following steps of:
S1: transducing at least one therapeutic RNA sequence together with at least one therapeutic RNA packaging sequence including an RNA binding protein sequence for an RNA binding protein into progenitors of megakaryocytes and/or CD34-expressing hematopoietic stem and progenitor cells (HSPC), immature megakaryocytes, and/or megakaryocytes, using a DNA-based vector, the therapeutic RNA sequence and/or packaging sequence encoding at least one therapeutic RNA product;
S2: activating expression of the therapeutic RNA sequence and/or the therapeutic RNA packaging sequence, and
S3: generating platelets from mature megakaryocytes derived from the transduced progenitors of megakaryocytes and/or from the transduced CD34-expressing hematopoietic stem and progenitor cells (HSPC), and/or from the transduced immature megakaryocytes and/or from the transduced megakaryocytes,
wherein said expression activation step is regulated to occur during maturation towards megakaryocytes and/or platelet formation;
wherein the generated platelet vehicles are functionally reactive to activation with specific agonists;
wherein the generated platelet vehicles are activated by extracellular signals, and
wherein the generated platelet vehicles contain the at least one therapeutic RNA product.

2. The method of claim 1, wherein the RNA binding protein is one among HNRNPA2B1, HNRNPC1, RBMX, HNRNPH1, HNRNPK, HNRNPQ, YBOX1, ELAV1, AGO2, IGF2BP1, MEX3C, ANXA2, PDC6I, NUCL, FUS, MVP, LIN28A, SRP14, SRP09, QKI, and TERT.

3. The method of claim 1, wherein the extracellular signals include a protein expressed on the surface of a cell, paracrine signals, and/or autocrine signals.

4. The method of claim 1 wherein the therapeutic RNA sequence encodes for at least one messenger RNA (mRNA), small interfering RNA (siRNA), short hairpin RNA (shRNA), long non-coding RNA (lncRNA), micro RNA (miRNA), or small activating RNA (saRNA).

5. The method of claim 1, wherein the therapeutic RNA sequence is single or in tandem for multiplexing.

6. The method of claim 1, wherein the therapeutic RNA sequence is adapted for:
targeting at least one among breast cancer and/or ovarian cancer, and/or siRNA for BRCA2 mutant, and/or
inhibition of a member of alt-NHEJ pathway, and/or
activation of a member of pro-apoptotic family BH3-only protein by saRNA.

7. The method of claim 1, wherein the therapeutic RNA sequence contains a specific protein binding motif or an EXOmotif comprising GAGAG or GGAG.

8. The method of claim 7, wherein the RNA binding protein is the protein counterpart of GAGAG or GGAG.

9. The method of claim 1, wherein expression of the therapeutic RNA and the therapeutic RNA packaging sequence is driven by a megakaryocyte specific promoter.

10. The method of claim 1, wherein the DNA-based vector further comprises at least one coding sequence for a chimeric or wild type activator receptor protein from the triggering receptor expressed on myeloid cells-like (TREM) family and/or from the Notch family with a wild type or a modified extracellular sequence.

11. The method of claim 10, wherein the modified extracellular domain of the chimeric activator receptor protein consists of or comprises a single-chain variable fragment (scFv) protein, a receptor ligand, a full receptor protein or an extracellular protein domain of a receptor, a nanobody, a designed ankyrin repeat proteins (DARPins), or adapter chimeric antigen receptor (adCAR).

12. The method of claim 11, wherein the chimeric activator receptor protein is constitutively expressed.

13. A therapeutic RNA platelet vehicle engineered through the method of claim 1, expressing at least one therapeutic coding sequence and/or at least one coding sequence for a receptor.

14. Use of the therapeutic RNA platelet vehicle of claim 13 for the treatment of a disease.

15. Use of the therapeutic RNA platelet vehicle of claim 13 for the treatment of at least one among cancer, acute inflammation, chronic inflammation, bleeding disorder, coagulation disorder, and/or liver disease.

16. A pharmaceutical composition comprising therapeutic RNA platelet vehicles engineered through the method of claim 1, said therapeutic RNA platelet vehicles expressing at least one therapeutic sequence and/or at least one coding sequence for a receptor.

17. Use of the pharmaceutical composition of claim 16 for the treatment of a disease.

18. Use of the pharmaceutical composition of claim 16 for the treatment of at least one among cancer, acute inflammation, chronic inflammation, bleeding disorder, coagulation disorder, and/or liver disease.


SEQUENCE LISTING (10 sequences)

SEQ ID NO: 1-2 - skipped (2 entries)

SEQ ID NO: 3 - RNA, 81 nt, Homo sapiens
  source 1..81: mol_type = transcribed RNA
cttcaggaagctggtttcatatggtggtttagatttaaatagtgattgtctagcaccatttgaaatcagtgttcttggggg

SEQ ID NO: 4 - RNA, 86 nt, Homo sapiens
  source 1..86: mol_type = transcribed RNA
tgccagtctctaggtccctgagaccctttaacctgtgaggacatccagggtcacaggtgaggttcttgggagcctggcgtctggcc

SEQ ID NO: 5 - RNA, 60 nt, Synthetic construct
  source 1..60: mol_type = other RNA
gaatctttggagtacgctggatgttttggccactgactgacatccagcgctccaaagatt

SEQ ID NO: 6 - RNA, 58 nt, Synthetic construct
  source 1..58: mol_type = other RNA
gaaacctgaatctggagtacgttttggccactgactgacgtactccagattcaggttt

SEQ ID NO: 7 - RNA, 60 nt, Synthetic construct
  source 1..60: mol_type = other RNA
gagtaagtcaacttcaatgtcggttttggccactgactgaccgacattggttgacttact

SEQ ID NO: 8 - RNA, 60 nt, Synthetic construct
  source 1..60: mol_type = other RNA
gaaatgtactgcgcgtggagacgttttggccactgactgacgtctccacgcagtacattt

SEQ ID NO: 9 - RNA, 11 nt, Synthetic construct
  source 1..11: mol_type = other RNA
aagcacccgtt

SEQ ID NO: 10 - RNA, 12 nt, Synthetic construct
  source 1..12: mol_type = other RNA
  repeat_region 8: note = The N at position 8 may vary in length from 1 to 20 nucleotides
nactaayntaay